(19) United States
(12) Patent Application Publication
(10) Pub. No.: US 2026/0016386 A1
(43) Pub. Date: Jan. 15, 2026

(54) OFF-AXIS LOADING FIXTURE FOR TESTING SPINE BIOMECHANICS

(71) Applicants: Axel Colbert Moore, Newark, DE (US); Dawn Marie Elliott, Newark, DE (US); Raith Nowak, Newark, DE (US); Sean Magee, Newark, DE (US); Imani Carter, Newark, DE (US); Gabriela Carlisle, Newark, DE (US); Justin Bouyer, Newark, DE (US)

(72) Inventors: Axel Colbert Moore, Newark, DE (US); Dawn Marie Elliott, Newark, DE (US); Raith Nowak, Newark, DE (US); Sean Magee, Newark, DE (US); Imani Carter, Newark, DE (US); Gabriela Carlisle, Newark, DE (US); Justin Bouyer, Newark, DE (US)

(73) Assignee: University of Delaware, Newark, DE (US)

(21) Appl. No.: 18/875,252

(22) PCT Filed: Jun. 16, 2023
(86) PCT No.: PCT/US2023/025562

§ 371 Date: Dec. 16, 2024

Related U.S. Application Data
(60) Provisional application No. 63/353,108, filed on Jun. 17, 2022.

(51) Int. Cl. G01N 3/56; G01N 3/04; G01N 3/14; G01N 3/16; G01N 3/34

(52) U.S. Cl. CPC G01N 3/56; G01N 3/04; G01N 3/14; G01N 3/165; G01N 3/34; G01N 2203/0003; G01N 2203/0007; G01N 2203/0019; G01N 2203/0028; G01N 2203/0035; G01N 2203/0037; G01N 2203/0252

(57) ABSTRACT

A multi-axial spine testing system includes a physiological motion unit (PMU); a uni-axial test frame having an input; and a fixture configured to be attached to the test frame and facilitates a plurality of primary motions of one or more of the PMU and the fixture. The plurality of primary motions includes a linear motion of the input and a rotational movement of an output. The fixture has an upper assembly directly or indirectly coupled to the input. The upper assembly transfers the linear motion (T_{z}) of the actuator to the rotational movement (Rₓ) of the output. The upper assembly and lower assembly apply a bending moment (M_{X}) and an axial compression (F_{z}) to the PMU. An external transducer collects data related to or generated by at least the plurality of primary motions. The fixture simultaneously provides the bending motion and axial compression to the PMU.

### CROSS-REFERENCE TO RELATED APPLICATIONS

This application claims priority from U.S. Provisional Application Ser. No. 63/353,108, titled “Modification of Single Axis Test Frames to Include Bending and Quasi Static Compression,” filed Jun. 17, 2022, the entirety of which is incorporated herein by reference.

### BACKGROUND OF THE INVENTION

The spine is a multi-tissue musculoskeletal system that supports large multi-axial loads and motions during physiological activities. Spine disorders such as disc prolapse or herniation, spondylosis, and spinal stenosis are associated with pain and loss of mobility for more than 20% of the population older than 50 years of age. These disorders directly affect the structure and composition of the spine (e.g., extrusion of the nucleus pulposus). Ex vivo studies in cadaveric spines are often used to study how these structural and compositional changes impact function. Prior work has focused on loading along a single axis of interest (e.g., axial compression or pure moments), but, physiologically, the spine is rarely loaded along a single axis. Therefore, it is of interest to consider all six axes, as well as coupled loading when evaluating spine function.

Various methods for measuring multi-axial spine mechanics or loading systems can be categorized into one of the following: (1) hexapod or stewart platform, (2) multi-axis robotic arm, (3) modified commercial test frame, or (4) custom build. Option (4) custom build is the most common approach, but it tends to require extensive development time and mechatronics expertise (e.g., in design, machining, instrumentation, control theory, programming, validation, etc.).

Thus, it is of interest to develop improvements in multi-axial spine testing systems, particularly a multi-axial spine testing system mounted to a conventional uni-axial test frame and methods of use thereof.

### SUMMARY OF THE INVENTION

The drawbacks of conventional spine testing systems are addressed in many respects by devices, methods, and systems in accordance with the invention.

One aspect of the invention is a multi-axial spine testing system. The system comprises a physiological motion unit (PMU) having a first end and a second end opposite the first end. The system also includes a uni-axial test frame having an input comprising a linear actuator. They system has a fixture configured to be attached to the uni-axial test frame and configured to facilitate a plurality of primary motions. The plurality of primary motions has a linear motion of the input and a rotational movement of an output. The fixture includes an upper assembly and a lower assembly. The upper assembly is configured to be directly or indirectly coupled to the linear actuator and the lower assembly is configured to be directly or indirectly coupled to the upper assembly. The upper assembly is also configured to transfer the linear motion (T_{Z}) of the linear actuator to the rotational movement (R_{X}) of the output. The upper assembly and the lower assembly are configured to apply an axial compression (F_{Z}) and a bending moment (M_{X}) to the PMU. The fixture is configured to simultaneously provide the bending motion and axial compression to the PMU. The system also includes an external transducer connected to the PMU and configured to collect data related to or generated by at least the plurality of primary motions.

Another aspect of the invention is a fixture. The fixture is configured to be attached to a PMU and a uni-axial test frame having an input. The fixture comprises an upper assembly and lower assembly. The upper assembly is configured to be directly or indirectly coupled to the input. The upper assembly and the lower assembly are configured to apply an axial compression (F_{Z}) and a bending moment (M_{X}) to the PMU. The fixture is configured to simultaneously provide the bending motion and axial compression to the PMU.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that according to common practice, the various features of the drawings are not drawn to scale unless otherwise indicated. On the contrary, the dimensions of the various features may be expanded or reduced for clarity. Included in the drawings are the following figures:

FIG. 1 depicts a multi-axial spine testing system in accordance with an exemplary embodiment of the invention;

FIGS. 2A-2C depict exemplary forces and motions of components of the system of FIG. 1;

FIGS. 3A-3G depict exemplary forces and motions of an exemplary fixture in accordance with an exemplary embodiment of the invention;

FIGS. 4A-4B depict an exemplary operation of components of the system of FIG. 1 in accordance with performance tests;

FIGS. 5A-5D depict graphs comparing calculated and measured bending profiles in accordance with performance tests of components of the system of FIG. 1;

FIGS. 6A-6F depict graphs comparing observed forces and motions of the exemplary fixture of FIGS. 3A-3G in accordance with performance tests of components of the system of FIG. 1;

FIGS. 7A-7B depict graphs observed forces and motions of the exemplary fixture of FIGS. 3A-3G in accordance with performance tests of components of the system of FIG. 1;

FIGS. 8A-8B depict graphs showing observed results of a long-term or fatigue testing of the exemplary fixture of FIGS. 3A-3G in accordance with performance tests of components of the system of FIG. 1;

FIGS. 9A-9D depicts graphs showing observed results of bending tests of the exemplary fixture of FIGS. 3A-3G in accordance with performance tests of components of the system of FIG. 1;

FIGS. 10 and 11 depict graphs showing observed exemplary forces produced by the exemplary fixture of FIGS. 3A-3G in accordance with performance tests of components of the system of FIG. 1;

FIG. 12A depicts an exemplary fixture illustrating exemplary alternate embodiments of the invention;

FIG. 12B depicts graphs showing observed comparative exemplary secondary moments produced by the exemplary fixture embodiments of FIG. 12A in accordance with performance tests of components of the system;

FIG. 13 depicts a graph showing observed exemplary cycles of flexion-extension applied to the exemplary fixture of FIGS. 3A-3G in accordance with performance tests of components of the system of FIG. 1; and

FIG. 14 depicts a graph showing observed exemplary Compressive spring stiffness applied to the exemplary fixture of FIGS. 3A-3G in accordance with performance tests of components of the system of FIG. 1.

### DETAILED DESCRIPTION OF THE INVENTION

Aspects of this invention relate to multi-axial spine testing systems, particularly a multi-axial spine testing system mounted to a conventional uni-axial test frame and methods of use thereof.

Although the invention is illustrated and described herein with reference to specific embodiments, the invention is not intended to be limited to the details shown. Rather, various modifications may be made in the details within the scope and range of equivalents of the claims and without departing from the invention.

Additionally, various forms and embodiments of the invention are illustrated in the figures. It will be appreciated that the combination and arrangement of some or all features of any of the embodiments with other embodiments is specifically contemplated herein. Accordingly, this detailed disclosure expressly includes the specific embodiments illustrated herein, combinations and sub-combinations of features of the illustrated embodiments, and variations of the illustrated embodiments.

Various terms are used throughout the disclosure to describe the physical shape or arrangement of features. A number of these terms are used to describe features that conform to a cylindrical or generally cylindrical geometry characterized by a radius and a center axis perpendicular to the radius. Unless a different meaning is specified, the terms are given the following meanings. The terms “longitudinal”, “longitudinally”, “axial” and “axially” refer to a direction, dimension or orientation that is parallel to a center axis. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation.

Terms concerning attachments, coupling, engagement, and the like, such as “mounted,” “coupled,” “engaged,” “connected” and “interconnected,” refer to a relationship wherein structures are secured or attached to one another either directly, or indirectly through intervening structures, as well as both movable or rigid attachments or relationships, unless expressly described otherwise.

Although the multi-axial spine testing systems discussed below and throughout the specification are described in the context of biomechanical testing of a functional spinal unit (FSU), one skilled in the art would understand that other types of similar testing and similar specimens (e.g. body segments which can be modified by actions of various forces) are applicable. For example, similar specimens include a knee joint, a hip joint, and a temporomandibular joint. Other types of similar testing include fatigue testing of certain systems, such as a bike frame, gold clubs, hockey sticks, and other like systems or objects.

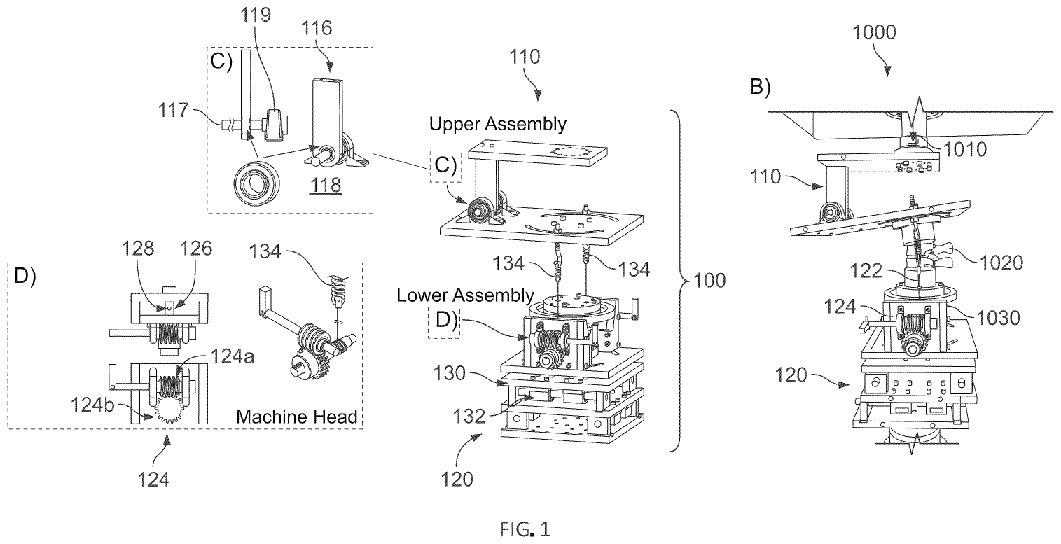

Referring generally to FIG. 1, a multi-axial spine testing system is disclosed. Generally, the multi-axial spine testing system 1000 includes a physiological motion unit (PMU), such as a functional spinal unit (FSU) 1020, a uni-axial test frame, and a fixture 100. Non-limiting examples of the test frame include conventional or existing uni-axial test frames, as designed and manufactured by Instron of Norwood, Massachusetts; TA Instruments of New Castle, Delaware; and MTS Systems. In an exemplary embodiment, the multi-axial spine testing system 1000 is configured to simultaneously provide bending (e.g. spinal flexion, spinal extension, spinal lateral bending, etc.) and axial compression to a specimen, such as FSU 1020, which comprises a single level human thoracic and/or lumbar FSU. Additional details of the multi-axial spine testing system 1000 and fixture 100 are discussed below.

As shown in FIG. 1, the multi-axial spine testing system 1000 includes a uni-axial test frame (not completely shown) having an input comprising a linear actuator 1010. In an exemplary embodiment, the test frame includes a TA Electroforce 3510 test frame, as designed and manufactured by TA Instruments of New Castle, Delaware. The system 1000 also includes a fixture, such as fixture 100, configured to be directly or indirectly attached to the uni-axial test frame. To facilitate this attachment, fixture 100 includes attachment surfaces, which can be modified based on the type of test frame used in system 1000. In a non-limiting example, attachment surfaces comprise bolt patterns which can be modified based on corresponding attachment surfaces provided by the type of test frame used in system 1000. Connected to the FSU is an external transducer 1030 (discussed further below), which is configured to collect data related to or generated by at least the fixture 100, such as the plurality of primary motions, primary forces, primary moments, or combinations thereof (discussed further below).

In an exemplary embodiment, the fixture 100, or off-axis loading fixture (OLaF™) assembly 100, includes an upper assembly 110 and a lower assembly 120, as shown in at least FIGS. 1, 2A-2C, and 3A-3G. The upper assembly 110 is configured to be indirectly or directly coupled to the lower assembly 120. The upper assembly 110 includes a top plate 112 defining the attachment surface configured to mate with a corresponding attachment surface of the test frame. The top plate or surface 112 generally defines a planar surface and has a generally rectangular geometry. However, one skilled in the art would understand from the description herein that other shapes, sizes, and configurations of the components of upper assembly 110 and fixture 100 are possible based on the specific configurations desired and/or other components of system 1000, such as the corresponding test frame. Further, the upper assembly 110 includes a pivoting platform 114 and a connector 116 connecting the top surface 110 to the pivoting platform 114. The pivoting platform defines a planar surface and has a generally rectangular geometry. In an exemplary embodiment, connector 116 includes a shaft connecting the pivoting platform 114 and the top surface 110, and a singular spherical bearins 118, which is mounted on the pivoting platform 114, such that the shaft 117 is positioned therebetween. Shaft 117 may further be disposed within one or more line or shaft (ball) bearings 119. In a non-limiting example, the spherical bearing 118 is self-lubricated and has a rated load capacity of 88.3 kN. The shaft and a pair of line or shaft bearings 119 define a line of upper assembly 110 that may move in a path relative to upper assembly 110 of fixture 100. To achieve this, the spherical bearing 118 is configured to provide three rotational degrees of freedom in all three axes (X, Y, Z).

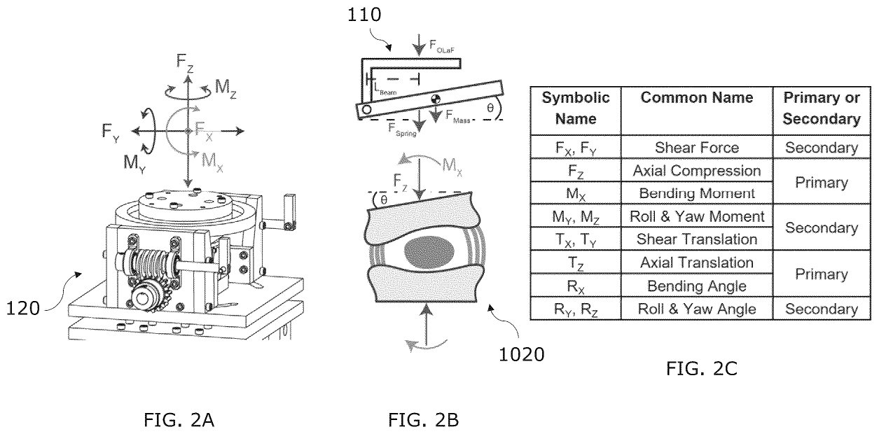

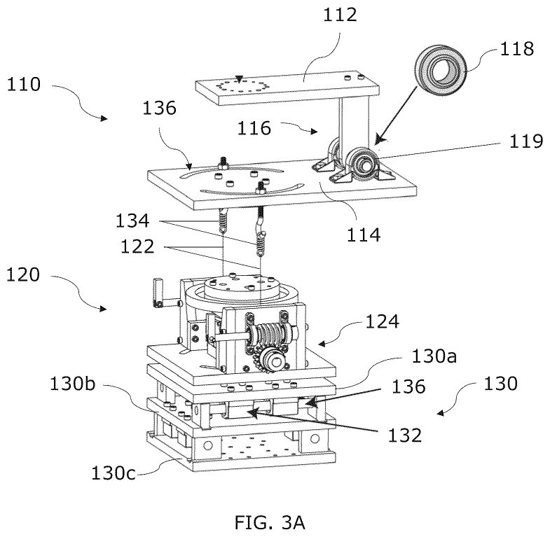

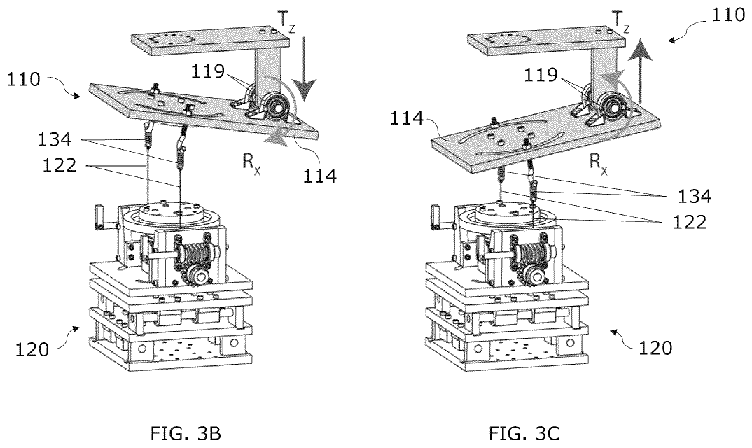

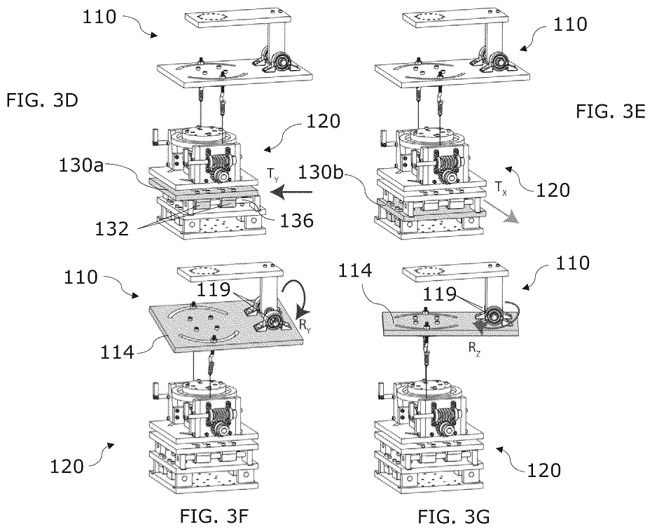

In this way, the fixture 100 is configured to facilitate a plurality of primary motions of one or more of the fixture 100 and the FSU 1020. In an exemplary embodiment, the fixture 100 is configured to facilitate a plurality of primary motions of the upper assembly 110. FIGS. 2A-2C shows a plurality of primary motions, forces, and moments and a plurality of secondary motions, forces, and moments produced by the fixture 100. In particular, FIGS. 2A-2C shows machine coordinates for forces (F), moments (M), translations (T) and rotations (R). In doing so, the multi-axial spine testing system 1000 simultaneously provides bending (e.g. flexion, extension, lateral bending, etc.) and axial compression to the FSU 1020. In an exemplary embodiment, as shown in FIGS. 2A-2C and 3A-3G, the plurality of primary motions includes a linear motion of the input 1010 and a rotational movement of an output. To achieve this, the upper assembly 110 is configured to be directly or indirectly coupled to the linear actuator 1010. Additionally, or optionally, the top surface 110 is configured to be directly or indirectly coupled to the linear actuator 1010. On the other hand, the pivoting platform 114 is configured to be attached to (or indirectly or directly coupled to) a first end of the FSU 1020. Thus, the FSU 1020 is placed or is positionable between the pivoting platform 114 of the upper assembly 110 and the top surface 110 of the upper assembly 110.

In this configuration, as shown in FIGS. 3A and 3B, the upper assembly 110 provides the offset loading that transforms or transfers the linear motion (T_{Z}) of the linear actuator 1010 to the rotational movement (R_{X}) of the output. In this way, the upper assembly 110 and lower assembly 120 is configured to provide a bending moment (M_{X}) to the FSU 1020 and an axial compression (F_{Z}) to the FSU 1020. In an exemplary embodiment, the fixture 100 is configured to simultaneously provide the bending moment (M_{X}) and axial compression (F_{Z}) to the FSU 1020. More particularly, the upper assembly 110 is configured to transfer the linear motion (T_{Z}) of the linear actuator 1010 to the rotational movement (R_{X}) of the spherical bearing 118. Thus, in an exemplary embodiment, the plurality of primary motions comprises the linear motion (T_{Z}) of the linear actuator 1010, the rotational movement (R_{X}) of the output, the bending moment (M_{X}) of the FSU 1020, and the axial compression (F_{Z}) applied to the FSU 1020. As used herein and throughout the specification, the bending moment (M_{X}) comprises one or more motions of the FSU 1020 corresponding to a spinal extension, a spinal flexion, a lateral flexion, or a combination thereof. The external transducer 1030, such as a six-axis load cell (e.g. an AMTI® MC3A-500 multi-axis transducer, from Advanced Mechanical Technology, Inc., Watertown, MA (USA), is configured quantify at least the forces and moments produced by the fixture 100 or applied to the FSU 1020, as illustrated in FIGS. 2A-2C. To facilitate this, the transducer 1030 is connected or placed adjacent the FSU 1020 (e.g. below the FSU 1020 and adjacent the lower assembly 120). While described herein in connection with an embodiment utilizing a transducer having 6 degrees of freedom (6DOF), it should be understood that the invention is not limited to any particular number of axes to be measured, degrees of freedom, or measurement technology.

As shown in FIG. 2B, bending is achieved by attaching the fixed end of a cantilevered beam (e.g. pivoting platform 114) to the FSU 1020 while the ‘free-end’ of the beam is vertically displaced. Under the assumption that the cantilevered beam is rigid and the fixed end is attached to an elastic foundation (e.g. FSU 1020), the vertical displacement (T_{Z}) required to achieve a given rotation is calculated using Equation 1 (Eq 1), listed below. In system 1000 comprising fixture 100, the bending moment (M_{X}) applied by the system 1000 is more than 2 orders of magnitude stiffer (˜420 N·m/deg) than the disc (e.g., human lumbar spine ˜1 N·m/deg), such that all deformations are assumed to occur within the FSU 1020.

$${T}_{z} = {L}_{beam} \cdot sin θ \qquad Equation 1$$

The test frame's linear actuator 1010 is used to apply T_{Z}. In this way, the fixture 100's primary bending axis (R_{X}) is driven by translations along T_{Z} and provides passive control of all secondary axes. The force (F_{OLaF}) required to displace the cantilevered beam multiplied by the distance between the instantaneous centers of rotation (L_{Beam}) is the applied moment (M_{X}), as illustrated in FIG. 2B. Identification of the instantaneous center of rotation for the pinned joint (e.g. spherical bearing 118) does not change with bending so it is assumed that the instantaneous center of rotation for the FSU 1020 is the geometric center. However, this is not always true, such as when FSU 1020 is positioned or configured for motion corresponding to flexion or extension. Furthermore, the instantaneous center of rotation can change with bending angle. To approximate the angulation error, the typical L_{Beam}=159 mm and an uncertainty of ±5 mm is assumed. Given a target angulation of 4 deg, the real angle would lie between 3.88 and 4.13 deg, or a 3% error.

In an exemplary embodiment, as best illustrated in FIGS. 1 and 3A, the lower assembly 120 contains at least one gear assembly or machine heads 124. Gear assembly 124 is configured to apply a separate axial compressive force (F_{Z}) to the FSU 1020. To facilitate this, the gear assembly 124 comprises a worm gear 124a and at a spur gear 124b engaged with and coupled to each other. Additionally, one end portion of each cable 122 is attached to the upper assembly 110 and another opposite end portion of each cable 122 is attached to the lower assembly 120. In particular, the one end portion of each cable 122 is attached to the pivoting platform 114 and the other opposite end portion of each cable 122 is attached to a drum 126 of the gear assembly 124 of the lower assembly 120. The drum 126 defines a hollow rod 128 through which the cable runs or passes through. Additionally, or optionally, each cable 122 of the lower assembly 120 is connected to the pivoting platform 114 via a tension spring 134, such that gear assembly 124 is configured to adjust a tension of the cable (F_{spring}). In operation, the drum 126 is rotated, thereby creating or adjusting the tension in each cable 122 that applies or contributes to the axial compressive force (F_{Z}) to the FSU 1020. In a non-limiting example, the gear assembly 124 (18:1 gear ratio) and each metal cable 122 with an inline tension spring 134 around the drum 126 can apply up to 925 N of tension to each side of the FSU 1020 for a combined maximum load of 1850 N. The FSU 1020 opposes the cable tension (F_{spring}) through compressive stress. Notably, as the FSU 1020 bends, the line of action for F_{spring} also changes with the FSU 1020. Although depicted in the exemplary embodiment with two cables 122 and a single gear assembly 124, it should be understood that the invention is not limited to any particular number of cables or gear assemblies, and that more or fewer of each may be present.

In an exemplary embodiment, with reference to FIGS. 2A-2C, the axial compression (M_{X}) is a sum of F_{spring}, an axial force (F_{OLaF}) required to produce the bending moment (F_{Z}) and a force applied by the pivoting platform 114 of the upper assembly 110 (Fₘₐₛₛ) (F_{Z}=F_{spring}+F_{OLaF}+F_{Mass}). Additionally, or optionally, the axial compression (F_{Z}) applied to the FSU 1020 is up to 750 N. The majority of the axial compression (F_{Z}) is contributed b F_{spring}. Regarding F_{OLaF}, a target angulation of ±4 deg, L_{Beam}=159 mm, and bending stiffness of 1 N·m/deg is assumed, so the estimated axial load variation is ±50 N. Finally, the pivoting platform 114 acts as a dead weight load (F_{Mass}) on the FSU 1020, including when the system 1000 is static (e.g. fixture and/or FSU 1020 is/are stationary), and no bending or spring tension is applied. In an exemplary embodiment, F_{Mass} is 21 N.

In operation, the plurality of primary motions (and the related primary forces and moments) facilitated by the fixture 100 can generate a plurality of secondary motions (and the related secondary forces and moments). Under ideal conditions (FSU 1020 geometry, material properties, alignment, high precision machining, etc.), all other secondary forces, moments, translations, and rotations would be nominal or zero. However, FSU 1020 inherently develops coupled motions and it is desirable to minimize secondary off-axis constraints (see FIGS. 2C and 3D-3G). In a non-limiting example, the plurality of secondary motions (and related secondary moments and forces) is different from the plurality of primary motions (and related primary moments and forces). As shown in FIGS. 2A-2C and FIGS. 3D-3G, the plurality of secondary motions comprises one or more of shear displacement along the X-axis and Y-axis (Tₓ, T_{y}) and/or the roll and yaw angle (R_{y}, R_{z}), and the plurality of secondary moments and forces includes shear force (Fₓ, F_{y}), and/or roll and yaw moment (M_{y}, M_{z}), all of which are produced by the fixture 100 in providing the bending moment (Mₓ). Additionally, or optionally, fixture 100 is configured to facilitate the plurality of primary motions while simultaneously minimizing a plurality of secondary moments and forces. To achieve this, lower assembly 120 further comprises a sliding stage 130 positionable on a plurality of linear rails 136 and a plurality of linear bearings 132 for minimizing the plurality of secondary moments and forces. In a non-limiting example, the plurality of secondary moments and forces is minimized below a predetermined threshold, such as a range of up to 5%.

In an exemplary embodiment, and with reference to FIGS. 3D-3E, the X-Y sliding stage 130 is positionable on four linear rails 136, such as 9338T53 linear rails from McMaster Carr of Cleveland, Ohio, with a combined load capacity of 1960 N and travel range of ±25.4 mm. The travel range is sufficient to permit the coupled shear translation that occur in biomechanical testing of a specimen, such as FSU 1020. Additionally or optionally, the sliding stage 130 comprises a plurality of plates, including a top plate 130a, a bottom plate 130c, and an intermediary plate 130b positionable between the top plate 130a and the bottom plate 130c (FIG. 3A). Additionally, or optionally, the spherical bearing 118, such as a model 63195K16 spherical bearing from McMaster Carr of Cleveland, Ohio, allows for rotations up to ±19 deg in R_{Y} and R_{Z} (FIGS. 3F and 3G). These secondary rotational degrees of freedom are sufficient to allow for coupled rotations that occur in during testing of FSU 1020.

### Example

The co-inventors assessed feasibility and functionality of the components of the devices, methods, and systems as disclosed herein, as well as verified any updates or improvements made. The prototype devices, methods, and systems were subjected to various performance tests as detailed herein.

The materials used to construct system embodiment 1000 and prototype fixture 100 are detailed in Table 1 below. Components, part numbers, and quantity are listed below, but the invention is not limited to any particular quantity, type, materials, or construction of parts or components.

| | | |
|---|---|---|
| Part Number | Description | Qty |

Upper Assembly

| | | |
|---|---|---|
| Moment Arm Flat Plate | 6061 Aluminum, plate connecting to small | 1 |
| | mounting plate and specimen | |
| Spherical Bearing Moment | 6061 Aluminum, vertical off-set | 1 |
| Arm Dowel Plate | | |
| Moment Arm Dowel Rod | 6061 Aluminum, dowel connecting arm and | 1 |
| | plate | |
| 6462K16 | Stainless Steel, Shaft Collar (½″) | 2 |
| 92185A992 | 316 Stainless Steel, 10-32 Cap Screw (1″) | 2 |
| 63195K16 | Spherical Bearing (1″ OD, ½″ ID) | 1 |
| Top Compression Plate | 6061 Aluminum, connects with the specimen | 1 |
| | mounting plate and interacts with the moment | |
| | arm, winch system is directly connected to this | |
| | plate | |
| 5913K61 | Shielded Mounted Steel Ball Bearing, (½″) | 2 |
| 33045T78 | 304 Stainless Steel, 5/16″-18 Eye Bolt ( 11/8″) | 2 |
| 9491T15 | Screw-in Hook with One Hex Nut, 0.270″ | 2 |
| | Diameter, 1-¼″ Projection, Packs of 10 | |

Machine Head/Gear Assembly

| | | |
|---|---|---|
| Winch Back Plate | 6061 Aluminum, winch back plate | 2 |
| Winch Base Plate | 6061 Aluminum, base plate of housing | 2 |
| Winch Front Plate | 6061 Aluminum, front plate of housing | 2 |
| Winch Side Plates | 6061 Aluminum, side plates of housing | 4 |
| Winch Worm Shaft | 303 Stainless Steel, ½″ nominal diameter | 2 |
| Winch Main Shaft | 303 Stainless Steel, ⅝″ Nominal Diameter | 2 |
| Winch Handle | 6061 Aluminum, allows for loosening and | 2 |
| | tightening of compression system | |
| Winch Handle Pin | 6061 Aluminum, connects the winch handle | 2 |
| | together | |
| 57545K511 | Cast Iron, Gear (½″) Shaft Diameter | 2 |
| 57545K527 | 1144 Carbon Steel, Keyed Worm Gear (½″) | 2 |
| | Shaft Diameter | |
| Machine Key | Worm Gear, and Main Gear Keys, ⅛″ Thick | 4 |
| 2867T49 | Aluminum-Bronze, Sleeve Bearing (⅜″) | 2 |
| 2867T54 | Aluminum-Bronze, Sleeve Bearing (½″) | 2 |
| Worm Shaft Spacers | Aluminum ½″ ID Spacers | 4 |
| 8600N5 | Aluminum Mounted Ball Bearing, (⅜″) | 4 |
| 6462K16 | Stainless Steel, Shaft Collar (½″) | 2 |
| 6462K14 | Stainless Steel, Shaft Collar (⅜″) | 6 |
| 92185A991 | 316 Stainless Steel, 10-32 Cap Screw (¾″) | 28 |
| 93190A587 | 316 Stainless Steel, 5/16″-18 Hex Head ( 11/2″) | 4 |
| 91343A200 | 316 Stainless Steel, 5/16″-18 Flanged Serrated | 4 |
| | Nut | |

Compression System/Lower Assembly

| | | |
|---|---|---|
| Bottom Compression Plate | 6061 Aluminum, mounting plate between top | 1 |
| | sliding base and load cell | |
| 3450T82 | Galvanized Steel Wire Rope - Not for Lifting, | 25″ |
| | Braided, 7 × 7, 0.47″ Diameter, 25′ Length | |
| 1773N11 | Wire Rope Compression Sleeve and Thimble | 8 |
| | Kits-Not for Lifting, 3/64″ | |
| 3869T68 | Ball-with-Shank-End Roller Swage Wire Rope | 2 |
| | End Fittings-Not for Lifting, 3/64″ | |
| 8464N324 | Corrosion-Resistant Extension Springs with Loop | 2 |
| | Ends, 31.6 mm Length | |
| 93190A580 | Super-Corrosion-Resistant 316 Stainless Steel | 4 |
| | Hex Head Screws, ⅝″ Length, 5/16″-18 | |
| 3450T28 | Stainless Steel, Braided (⅛″) 25 ft | 1 |
| 3755T15 | Wire Crimping Sleeves for ⅛″ Wire | 8 |
| 8464N492 | Tensile Springs with 200 lbs Capacity | 2 |
| 8464N324 | Tensile Springs | 2 |
| 91343A200 | 316 Stainless Steel, 5/16″-18 Flanged Serrated | 2 |
| | Nut | |

FSU Specimen Mounting

| | | |
|---|---|---|
| Top Specimen Mounting | 6061 Aluminum, directly interfaces with | 1 |
| Plate | Specimen | |
| Bottom Specimen Mounting | 6061 Aluminum, interfaces with Reservoir | 1 |
| Plate | Interface Plate, and Specimen | |
| Reservoir Interface Plate | 6061 Aluminum, Interfaces Bottom Specimen | 1 |
| | Plate and Load Cell | |
| 92185A991 | 316 Stainless Steel, 10-32 Cap Screw (¾″) | 4 |
| 91251A540 | Alloy Steel ¼″-20 Cap Screw (¾″) | 4 |

Sliding Base (Stage and Linear Rails)

| | | |
|---|---|---|
| Sliding Base-Bottom Plate | Spacer Plate between the Top Sliding Base | 1 |
| Spacer Plate | Plate, and the Bottom Compression Plate | |
| Sliding Base Top Plate | 6061 Aluminum, Top Plate of Sliding Base | 1 |
| Sliding Base Center Plate | 6061 Aluminum, Middle Plate of sliding base, | 1 |
| | connecting the x and y sliding bearings | |
| Sliding Base Bottom Plate | 6061 Aluminum, Mounting plate between the | 1 |
| | sliding base and the load frame | |
| Sliding Base Rail Mount | 6061 Aluminum, allows for suspended mounting | 8 |
| | of the linear rails, providing room for the | |
| | bearings to fit and move freely | |
| 6112K103 | 12 mm diameter 1055 Carbon Steel, Linear | 4 |
| | Motion Shaft | |
| 9338T53 | Mounted Linear Ball Bearing, Steel Bearing w/ | 8 |
| | 6061 Aluminum Housing | |
| 91375A438 | ¼″ 10-32 Alloy Steel Cup-Point Set Screws | 8 |
| 92290A252 | 316 Stainless Steel, M5-0.8 Cap Screw (25 mm) | 32 |
| 92185A991 | 316 Stainless Steel, 10-32 Cap Screw (¾″) | 8 |
| 9506T6 | 12 mm Diameter Clamping Shaft Collar | 2 |

Miscellaneous

| | | |
|---|---|---|
| AMTI MC3A-500 Load Cell | 6 axis load cell for measuring forces and torques | 1 |
| 91251A539 | Alloy Steel ¼″-20 Cap Screw (⅝″) | 4 |
| Small Mounting Plate | 6061 Aluminum, Plate connects moment arm to | 2 |
| | actuator | |

The various clinical tests and certain parameters for each performance test are summarized in Table 2 below.

TABLE 2
List of Performance Tests. A nominal
load of 0 is ~21N due to F_{Mass}

| Test | Test | | Angle | Frequency | Cycles | Nominal |
|---|---|---|---|---|---|---|
| No. | Identity | Material | (deg) | (Hz) | (#) | Load (N) |
| 1 | Motion | Spring | ±4 | 0.5 | 5 | 0 |
| | Capture | | | | | |
| 2 | Axial Load | | | 0.5 | 5 | 0 vs 200 |
| 3 | X-Y Stage | | | 0.5 | 5 | 0 |
| | Constraints | | | | | |
| 4 | Cycling | | | 0.01 to 1.0 | 5 | 0 |
| | Frequency | | | | | |
| 5 | Test | | | 0.5 | 3600 | 200 |
| | Duration | | | | | |
| 6 | Flexion- | FSU | ±3 | 0.5 | 5 | 200 |
| | Extension | | | | | |
| 7 | Lateral | | ±4 | | | |
| | Bending | | | | | |
| 8 | Direc- | | ±4 | | | |
| | tionality | | | | | |
| | Testing | | | | | |

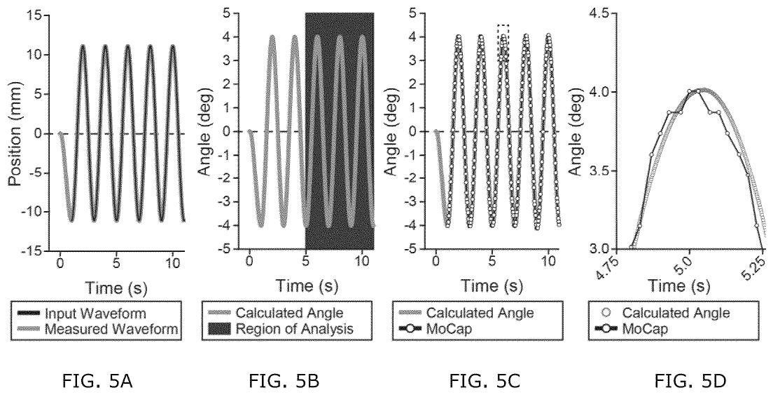

A range of frequencies and cycle numbers can be used to simulate various aspects of daily life (e.g., a range of daily tasks including lying supine, sitting, standing, walking, etc.). The frequency of 0.5 Hz was selected to demonstrate the capability of fixture 100 to perform bending tests at a moderate physiological speed (FIG. 5A). Furthermore, the disc achieves a steady-state-like performance by the 3^{rd} cycle; thus 5 cycles were performed and the last 3 cycles were used for analysis (FIG. 5B). A 200 N axial load was chosen to produce a physiologically relevant load in the human lumbar spine. A 200 N axial load on the human lumbar spine approximates a 0.23 MPa NP pressure, which represents physiological conditions such as lying supine.

### Procedure

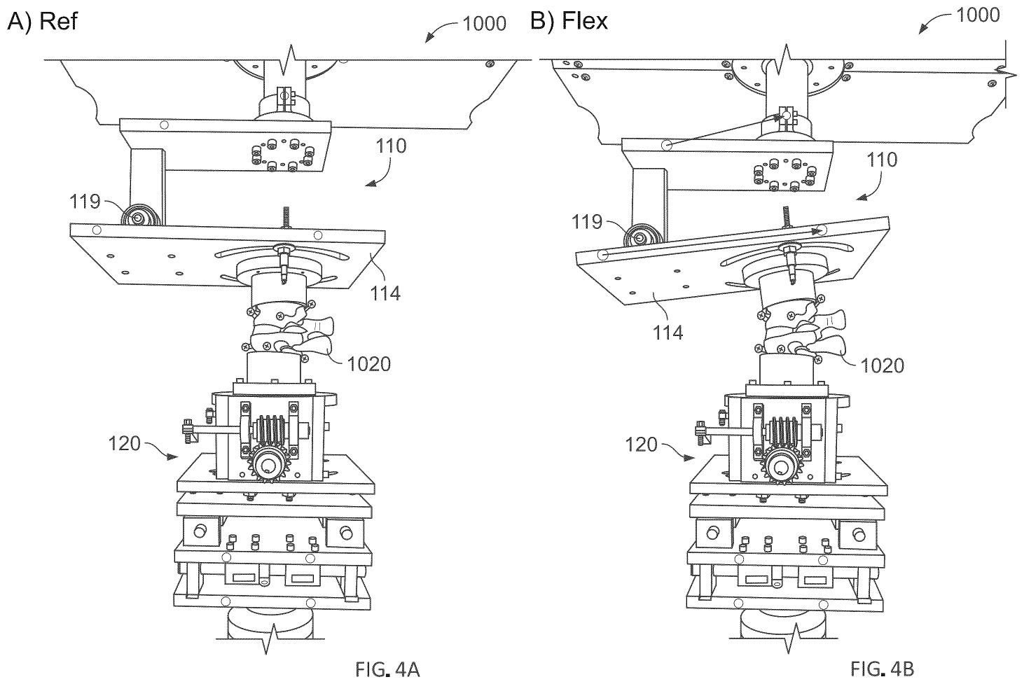

To assess the performance of prototype fixture 100 a standard testing protocol was used, as outlined below.
1. Attach prototype fixture 100 to the test frame (FIGS. 4A-4B)
2. If using a motion capture system-set up and calibrate
3. Define a bending profile, which includes a bending amplitude (R_{X}), frequency, and number of cycles (Table 2)
4. Calculate the axial translation (T_{Z}), speed, and duration necessary to achieve the desired bending dynamics or profile.
5. Input the desired waveform into the test frame's software (e.g. WinTest 7 for TA Electroforce, from TA instruments of New Castle, Delaware)
6. Zero the external transducer, such a six-axis load cell as described in more detail elsewhere herein.
7. Attach the specimen to the OLaF assembly in a neutral posture (FIG. 4A)
8. Apply the desired compressive load (F_{Z}) using the gear assembly or machine heads (Table 2) while maintaining a neutral FSU posture.
9. If using a motion capture system-start recording.
10. Start the experiment.

Following testing, the bending stiffness is calculated using a least squares linear regression to the last 3 cycles of the moment (M_{X}) versus angle (R_{X}) data. The reported bending stiffness values for the FSU should be taken with caution as the FSU displayed the expected nonlinear behavior. A linear regression was selected to detect relative changes rather than absolute values.

### Test Parameters

Transducer range, motion and load limits, accuracy, and noise for the test frame (TA Electroforce 3510), six-axis load cell (AMTI MC3A-500), and the OLaF™ assembly were established and are listed below in Table 3. All values are given as ±value (i.e., test frame T_{Z}=±25 mm). Bold values indicate operational limits defined by the test frame or load cell. Operational limits not defined by the test frame or load cell are based on the kinematics or manufactured components of the OLaF™ assembly (e.g., range of motion of X-Y stage and safe working load for cables) and are indicated by an ampersand (&). The calibrated accuracy is taken from the calibration report from the manufacturer or supplier of each component. Values experimentally determined by the authors are indicated by an asterisk (*). In principle, alternative test frames and load cells can be swapped into this table to define new operational limits.

TABLE 3
Test Parameters

| Parameter for | | | |
|---|---|---|---|
| Translation (T), | | | |
| Rotation (R), | | Six-Axis Load | |
| Force (F), and | | Cell/External | |
| Moment (M) | Test Frame | Transducer | OLaF/Fixture |
| Transducer | T_{Z} = 25 mm | F_{X}&F_{Y} = 1112N | T_{Z} = 25 mm |
| Range (±value) | | F_{Z} = 2224N | F_{X}&F_{Y} = 1112N |
| | | M_{X}&M_{Y} = 56 N · m | F_{Z} = 2224N |
| | | M_{Z} = 28 N · m | M_{X}&M_{Y} = 56 N · m |
| | | | M_{Z} = 28 N · m |
| Motion Limits | T_{Z} = 25 mm | No Moving Elements | ^{&}T_{X}&T_{Y} = 25.4 mm |
| (T & R) | | | T_{Z} = 25 mm |
| | | | ^{&}R_{X} = 30 deg |
| | | | ^{&}R_{Y}&R_{Z} = 19 deg |
| Load Limits | F_{X}&F_{Y} = 330N | See Transducer Range | F_{X}&F_{Y} = 330N |
| (F & M) | M_{X}&M_{Y} = 80 N · m | | ^{&}F_{Z} = 1850N |
| | M_{Z} = 100 N · m | | ^{&}M_{X}&M_{Y} = 31.8 N · m |
| | | | M_{Z} = 28 N · m |
| Calibrated | T_{Z} = 0.065 mm | F_{X}&F_{Y} = 2.5N | T_{Z} = 0.065 mm |
| Accuracy | | F_{Z} = 6.6N | F_{X}&F_{Y} = 2.5N |
| | | M_{X}&M_{Y} = 0.20 N · m | F_{Z} = 6.6N |
| | | M_{Z} = 0.12 N · m | M_{X}&M_{Y} = 0.20 N · m |
| | | | M_{Z} = 0.12 N · m |
| RMS Noise | Not Tested in | Not Tested in Isolation | *T_{Z} = 0.005 mm |
| | Isolation | | *F_{X}&F_{Y} = 0.08N |
| | | | *F_{Z} = 0.4N |
| | | | *M_{X}&M_{Y} = 0.003 N · m |
| | | | *M_{Z} = 0.002 N · m |

### Performance Test: Spring

For performance test nos. 1 to 5, a compression die spring was chosen to minimize viscoelastic effects. The spring (see Table 1) had a nominal spring rate of 280 N/mm. The spring was then potted in Ortho-Jet acrylic resin to facilitate mounting in fixture 100. The potting reduced the effective spring length and increased the axial spring stiffness to 430 N/mm. FIG. 14 shows that testing demonstrated a linear response up to 950 N of axial compression.

### Performance Test No. 1

With reference to FIGS. 5A-5D, the objective of Performance Test No. 1 was to demonstrate a near zero-cost system for motion capture and compare the bending angle between the calculated bending angle (Equation 1) and measured bending angle (Motion Capture). FIG. 5A shows that the data input to the test frame is shown a linear translation along the Z-axis (T_{Z}). The RMS error between the input and measured waveform is 0.22 mm. FIG. 5B shows that the linear translation is converted to an angular rotation (Calculated Angle) by the spherical bearing. The target waveform is 0.5 Hz, ±4 deg, for 5 cycles. The last 3 complete cycles were analyzed (blue region). FIG. 5C shows that motion capture (MoCap) data is postprocessed using Kinovea. The MoCap angle is compared to the calculated angle. The RMS error between the measured waveform and MoCap is 0.35 deg. FIG. 5D shows a magnified view from the peak of the 3rd cycle. The data demonstrates two shortcomings of the MoCap system: (1) slower frame rate that produces fewer data points and (2) lower resolution that produces step like behavior).

More specifically, the accuracy of the input waveform to the output response is established. Using Eq. 1 the desired bending (R_{X}) amplitude is transformed into an axial translation (T_{Z}). An example input waveform to achieve ±4 deg of bending is shown in FIG. 5A. It should be noted that the measured position (test frame displacement sensor) of the system differs slightly from the input waveform. The RMS error is ˜0.22 mm. Converting this translational error to a rotational error gives 0.08 deg. With the accuracy of the input waveform to output response confirmed, the next aim is to demonstrate the zero-cost motion capture system. To do this, a personal smartphone camera with a free motion analysis software (such as Kinovea) was used to quantify the dynamics of inventive fixture 100. The results of the motion capture are shown in FIGS. 5C and 5D and appear to be very well correlated. The RMS error between motion capture and the calculated bending angle was 0.35 deg (<4% error). The cause of this error likely stems from the slow frame rate and resolution in the motion capture system used. Other potential sources of error include non-planar motion (R_{Y} and R_{Z}), component tolerances (bearings), an incorrectly measured or changing center of rotation, and system compliance (beams and bolted connections). Despite this, a 4% error for a near zero-cost motion capture system is acceptable. Applications requiring more accuracy may use a higher fidelity motion capture system with fully automated point tracking.

### Performance Test Nos. 2 & 3

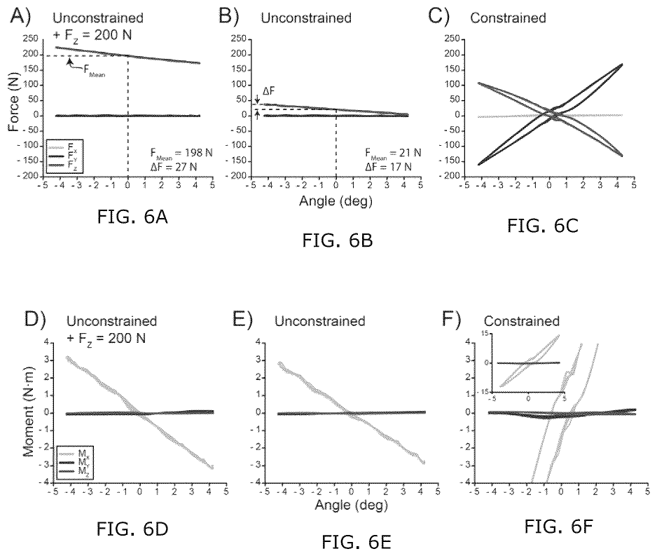

With reference to FIGS. 6A-6F, the objective of Performance Test Nos. 2 and 3 was to evaluate the effect of axial compression variability (due to F_{OLaF} and applied F_{spring}) and effect of the sliding stage or X-Y stage to eliminate or minimize secondary off-axis constraints.

Resulting forces (FIGS. 6A-6C) and moments (FIGS. 6D-6F) when testing with and without applying an additional compressive load through the machine head (F_{spring}), and with and without the X-Y stage to minimize the secondary constraints. As shown in FIGS. 6A and 6D, the fixture 100 is loaded to F_{Mean}˜200 N in F_{Z}. The X-Y stage is unconstrained. The bending stiffness is 0.76 N·m/deg. In contrast, FIGS. 6B and 6E show that the load imposed by the machine heads or gear assembly is removed, thereby producing F_{Mean}=21 N and a force variation (ΔF)±17 N. Under a stress-free reference configuration, F_{Mean}=F_{Mass}. Furthermore, ΔF=F_{OLaF} and is the offset force required to bend the FSU specimen. The bending stiffness is 0.67 N·m/deg. As shown in FIGS. 6C and 6F, the X-Y stage is constrained by shaft collars and forces all shear displacements to occur within the FSU specimen. The response of the FSU specimen is greatly affected by this constraint and violates a goal of minimizing secondary constraints.

As stated above, F_{Z} is a summation of three contributing forces (F_{OLaF}, F_{Mass}, F_{spring}). In the reference experiment, the machine heads or gear assembly is configured to yield a nominal F_{Z}=200 N and the X-Y stage is unconstrained in T_{X} and T_{Y} (FIGS. 6A and 6D). The calculated bending stiffness was 0.76 N·m/deg. The peak secondary shear and moment were 2.1 N and 0.1 N·m, which are <4% of the applied axial load (200 N) and bending moment amplitude (3.2 N·m). Next, the axial load from F_{Spring} is removed (FIG. 6B) and the experiment is repeated. FIGS. 6B and 6E demonstrate a similar moment-angle response with secondary forces and moments remaining negligible. The mean compression force, F_{Mean}=21 N, corresponds to the mass of the upper assembly (F_{Mass}). The change in force during loading, ΔF=17 N, corresponds to the F_{OLaF} required to achieve the desired rotation. Finally, the linear bearings were disabled using shaft collars to lock the X-Y stage, forcing all off-axis coupled shear motions to be carried by the specimen. This constrained system produces a greatly different response (FIG. 6C and FIG. 6F) with large F_{Z} and F_{Y} amplitudes >100 N and a bending moment that is ˜5×greater and in the opposite direction compared to the unconstrained conditions. These results demonstrate that the use of an unconstrained X-Y stage (FIGS. 3D and 3E) is a necessary design requirement for fixture 100 and system 1000 and the unconstrained X-Y stage configuration to eliminate secondary off-axis constraints are used for the remainder of the tests described herein.

### Performance Test No. 4

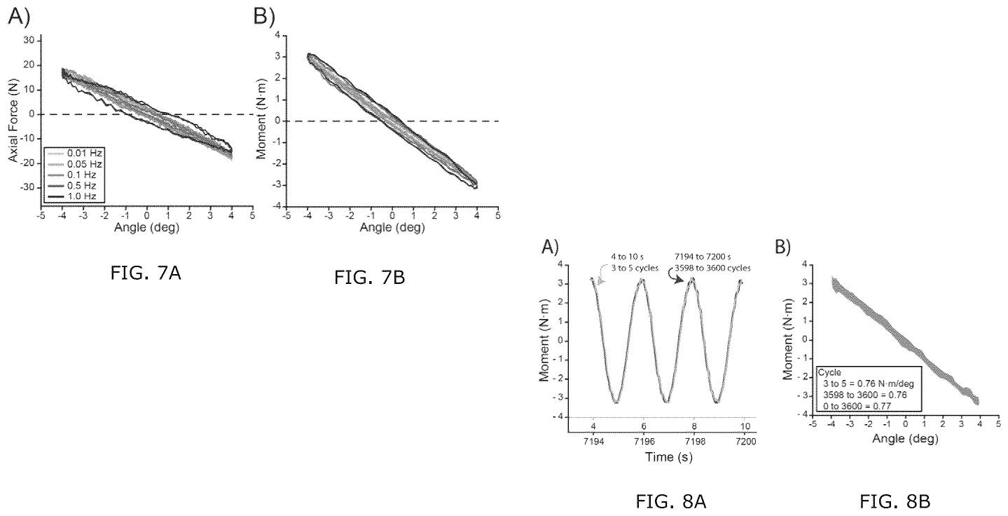

With reference to FIGS. 7A-7B, the objective of Performance Test No. 4 was to evaluate the effect of cycling frequency on the measured response. Specifically, the test aimed to evaluate when inertial effects from the moving mass begin to influence the response. FIG. 7A shows the resulting axial force and FIG. 7B shows the bending moment for a range of cycling frequencies (0.01 to 1.0 Hz). At 1 Hz, there is a small shift in the measured responses. The linear bending stiffness was 0.73 and 0.74 N·m/deg at 0.01 and 1.0 Hz respectively. Note that the relative axial force is shown by centering the mean force about 0 N.

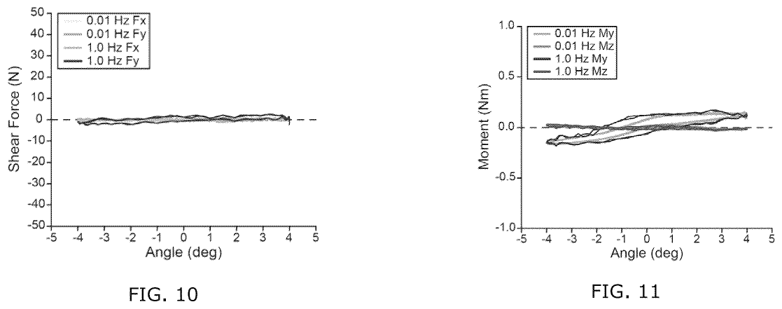

Specifically, the spring was used to isolate the inertia of fixture 100 by excluding the potential role of the rate-dependent behavior of the FSU specimen. The test frequency was varied over 2 orders of magnitude (0.01, 0.05, 0.1, 0.5, and 1.0 Hz). Between the slowest and the fastest frequency, the bending stiffness changed less than 2% (0.73 vs 0.74 N·m/deg), as shown in FIG. 7B. The secondary shears and moments were similarly insensitive to the cycling frequency, as shown in FIGS. 10 and 11. Namely, FIG. 10 shows shear forces for the slowest and fastest frequency tested on fixture 100. The shear forces are negligible compared to the axial load (FIG. 7A). FIG. 100 shows secondary moments for the slowest and fastest frequency tested on fixture 100. The secondary moments are negligible compared to the resulting bending moment (approximately ±3 N·m), as shown in FIG. 7B.

The data demonstrates that under this range of loading frequencies and amplitude, there is a negligible inertial effect.

### Performance Test No. 5

With reference to FIGS. 8A-8B, the objective of Performance Test No. 5 was to determine the stability of the system and the potential for long-term or fatigue testing. FIG. 8A shows the compression spring was cycled 3600 times at 0.5 Hz at ±4 deg. FIG. 8B shows the bending stiffnesses (0.76, 0.76, and 0.77 N·m/deg) are calculated using linear regressions for cycles 3 to 5, 3598 to 3600, and 0 to 3600. The nearly constant bending stiffness demonstrates the stability of fixture 100.

In particular, fixture 100 was ran for 2 hours (3600 cycles) to determine the stability of the system and the potential for long-term or fatigue testing. This test was performed on the compression spring to again remove any viscoelastic effects. The response of cycles 3-5 and 3598 to 3600 are shown in FIG. 8A. The moment versus time traces (FIG. 8B) are nearly identical despite being measured nearly 2 hr apart. The stability of the system is well suited for longer duration testing up to at least 2 hr.

### Performance Test: FSU

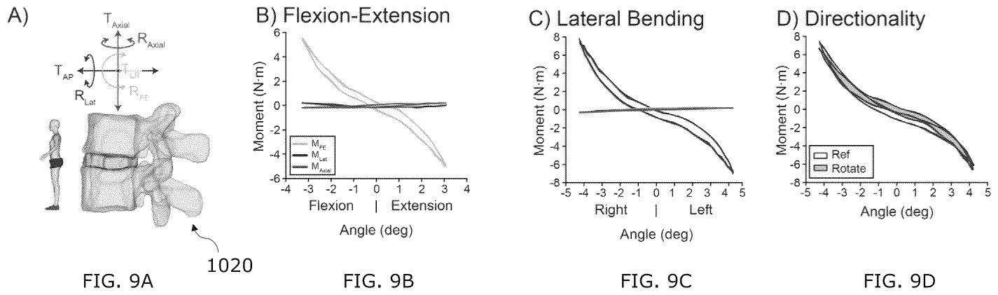

As stated above, the fixture 100 was designed for spine biomechanics testing. Therefore, a human donor FSU (male, 60 years of age, L1-L2) was used as a representative specimen. The whole spine was stored at −20° C. On the day of dissection, the specimen was defrosted in a vacuum sealed bag and submerged in a 27° C. water bath for 2 hr. The surrounding soft tissue and posterior elements were resected. The specimen was aligned (FIG. 9A) and potted in acrylic resin (such as an Ortho-Jet™ resin from Lang Dental Manufacturing Company, Inc. of Wheeling, Illinois) under fluoroscopic guidance. Saline-soaked gauze was wrapped around the disc during potting to maintain hydration. The potted FSU was then submerged in PBS at 4° C. with a 55.6 N static load for 19 hr to reach a steady state level of hydration and avoid supraphysiologic hydration.

### Performance Test Nos. 6 and 7

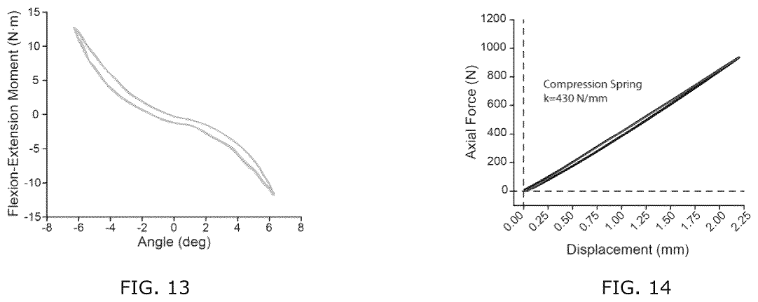

With reference to FIGS. 9A-9D, the objective of Performance Test Nos. 6 and 7 was to evaluate the FSU in flexion-extension and lateral bending. FIG. 9A shows an anatomical coordinate system showing Translations (T) and Rotations (R) of the human L1-L2 FSU, as described above. Anatomical Forces (F) and Moments (M) also follow this same coordinate system. FIG. 9B shows characteristics of flexion-extension the human L1-L2 FSU and FIG. 9C shows characteristics of lateral bending of the same human L1-L2 FSU specimen (see Table 1 for experimental conditions). FIG. 9D shows characterizes of lateral bending of the same human L1-L2 FSU specimen after removing the specimen and rotating it 180 deg about the Z-axis. A similar moment-angle response was observed. The moment-angle response has the expected shape and peak values based on existing literature. Furthermore, the non-linearity and hysteresis observed is also expected. In agreement with prior work, cycles 3 to 5 produce a consistent overlapping response demonstrating a steady state performance for short duration testing. The secondary forces and moments (not shown) remained below the 5% threshold. Further, FIG. 13 shows characteristics of the human lumbar FSU specimen, as it was taken through 5 cycles of flexion-extension at #6 deg.

### Performance Test No. 8

The objective of Performance Test No. 8 was to evaluate the impact of this load variation since axial load has been shown to influence the stiffness of human FSUs. The load that drives bending (F_{OLaF}) produces an unbalanced load with a greater F_{Z} in one direction of bending than the other (FIG. 6A). After testing the FSU in the reference position for lateral bending (FIG. 9C), the FSU specimen was removed then rotated 180 deg. The results are shown in FIG. 9D and demonstrate minor differences between the two configurations (Reference=1.36 versus Rotate=1.32 N·m/deg, a minimal 3% change).

### Performance Test: Sliding Stage (X-Y Stage)

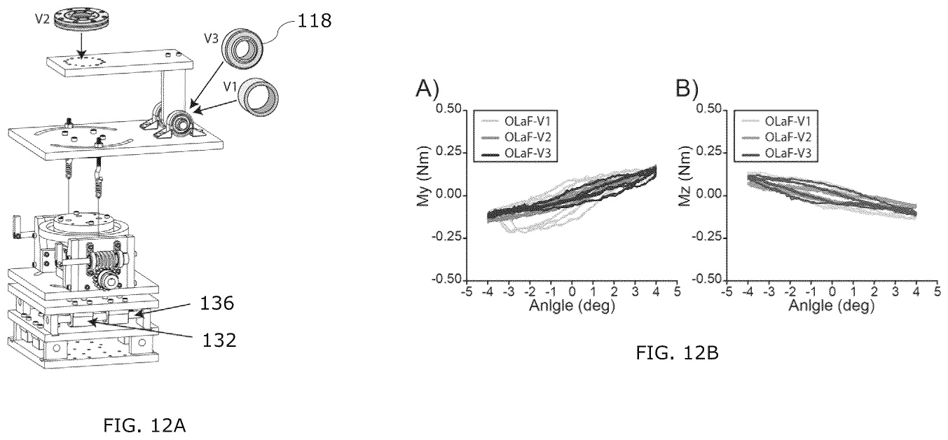

With reference to FIGS. 3A and 12A-12B, the X-Y table uses 8 linear bearings (4 per axis) riding on hardened rails (2 per axis). The X-Y table provides near frictionless shear translations of the specimen. One embodiment of the fixture (referred to herein as OLaF-V1 (V1)) features a sleeve bearing at the hinge. Another embodiment of the fixture (referred to herein as OLaF-V2 (V2)), features a sleeve bearing in addition to a thrust bearing between the OLaF™ assembly and the test frame. In another embodiment of the figures (referred to herein as OLaF-V3 (V3)), replaces the sleeve bearing with a spherical bearing, which also eliminates the need for the thrust bearing. The invention is not limited to any particular type or arrangement of bearings. Unless otherwise specified, the embodiment of the fixture primarily referred to herein comprises V3.

As shown in FIGS. 12A-12B, in V1, secondary rotations are constrained due to the use of a sleeve bearing. Machining tolerances, potting and specimen misalignment, specimen asymmetry, and material inhomogeneities are just a few of the conditions that can lead to secondary moments (M_{Y} and M_{Z}). Based on existing literature, it is known that flexion-extension and lateral bending lead to coupled rotations in the secondary axes. Other variations of fixtures were designed to minimize the potential for these secondary loads. In V2, a thrust bearing was added between the fixture and the test frame. The thrust bearing allows for unrestricted motion in R_{Z}. In V3, the sleeve bearing was replaced with a spherical bearing. The spherical bearing allows free rotations in R_{Y} and R_{Z} between ±19 deg.

Secondary moments in (A) Y and (B) Z for different configurations of a fixture (OLaF-V1, OLaF-V2, and OLaF-V3) are shown in FIG. 12B. The small spring specimen was oscillated between ±4 deg at 0.5 Hz for 5 cycles. The experiment was repeated 3 times for each configuration (3 configurations×3 repeats=9 experiments shown). In sum, the secondary moments for all configurations are small when compared to the primary bending moment of approximately ±3 N·m. V1 experienced the largest secondary moments. V2 and V3 are comparable in their secondary moments.

### Performance Test Nos. 1-8 Conclusion

The performance tests show that fixture 100 provides combined bending (demonstrated up to 6 deg (FIG. 13) and 1 Hz (FIG. 7A-7B)) and compression (demonstrated up to 950 N (FIG. 14) and capable of 1850 N (Table 1)) to physiological levels. Through a series of performance tests, fixture 100 and system 1000 provides compression and bending while minimizing secondary off-axis loads to less than 5%, has minimal inertial effects up to 1 Hz, and can be used for long duration testing (2 hr of runtime, as shown in FIG. 8). In addition to testing with a linear compression spring, fixture 100 was assessed for use with a human FSU. The human FSU specimen was tested in flexion-extension and lateral bending. The functional response of the FSU was conserved when the specimen orientation was rotated 180 deg.

Thus, a system 1000 including fixture 100 is feasible for achieving compression and bending profiles and the fixture 100 can be easily mounted to and removed from common uniaxial test frames. Furthermore, fixture 100 is configured to be controlled using the same program or similar programs as the test frame and the six-axis load cell data is collected using the software that is available, thereby minimizing technical challenges associated with developing custom software. Finally, a near zero-cost motion capture system, while not required, can be implemented using a smartphone camera and Kinovea (a free motion analysis software).

Additionally, fixture 100 is suited to higher axial loads which are more representative of sitting, standing, and walking, rather than lying supine. Further, as stated above, bending in fixture 100 is controlled and calculated from the displacement of the linear actuator. This calculation does not account for specimen compliance, creep, misalignment with the instantaneous center of rotation, changing instantaneous center of rotation, or slip at the mounting or potting interface. Nevertheless, good agreement was found using system 1000 and fixture 100 (4% error). Implementing a higher fidelity motion capture system could reduce sources of error. Finally, fixture 100 is configured to evaluate one axis of bending and compression at a time, such that the FSU specimen must be rotated for sequential testing of flexion-extension and then lateral bending. Torsion is another desirable loading modality for spine biomechanics research. However, conventional uniaxial test frames have attachments that enable compression+torsion testing.

### Performance Test: Motion Capture

With reference to FIGS. 4A-4B, the FSU and the fixture is a reference or neutral position (FIG. 4A) and the FSU is placed in flexion (FIG. 4B). In this performance test, the measured axial translation (T_{Z}) of the upper assembly to calculate FSU bending was assessed. An additional or alternative method for kinematic analysis includes use of a smartphone video recorder and Kinovea, a free and open-source motion analysis software. The smartphone used in this work was an iphone 13. The smartphone was mounted to a tripod and recorded high definition (1080p) video at 30 frames per second. As shown in FIG. 4A, a number of (9) red markers were placed on the fixture and captured within the video frame. In addition to the markers on the fixture, an index card aligned with the image plane was used to calibrate the image perspective. This calibration is a built-in function for Kinovea.

The video data was imported into Kinovea and each of the points of interest was identified and automatically tracked through the experiment. Position data was exported and analyzed using a custom MATLAB® script to calculate displacements (T_{Y}, T_{Z}) and rotation (R_{X}) as a function of time. The motion capture results were compared to the angle calculated based on the measured axial translation of the linear actuator. This motion analysis is limited to the plane of interest and only provides 2D information.

### Performance Test: System Noise and Error

System noise and error were calculated using the root mean square (RMS) method as shown in Equation 2 (Eq 2):

$$RMS = \sqrt{\frac{{\sum }_{i}^{n} {( {x}_{i} - \overline{x} )}^{2}}{n}} , \qquad Equation 2$$

where (xᵢ) are individual measures, (x̅) is the mean signal, and (n) is the number of measures.

The transducer noise was quantified by attaching the fixture to the test frame and commanded the linear actuator to hold a static position (T_{Z}). The data from the transducers (position sensor and load cell) are collected and used to calculate the RMS noise. The noise was found to be less than the calibrated accuracy of each component (see Table 1 above). This test demonstrated that the fixture does not compromise component accuracy. Furthermore, the fixture was cycled 5 times between ±4 deg against a near frictionless pivot. The load cell registered 0.6 N of noise in F_{Z} and 0.01 N·m of noise in M_{X}. This was less than the calibrated accuracy of the transducers and demonstrates that the dynamics of exemplary fixture did not compromise the accuracy of the system 1000.

Although the invention is illustrated and described herein with reference to specific embodiments, the invention is not intended to be limited to the details shown. Rather, various modifications may be made in the details within the scope and range of equivalents of the claims and without departing from the invention.

1.-5. (canceled)

6. A multi-axis test system, the system comprising:
a physiological motion unit (PMU) having a first end and a second end opposite the first end;
a uni-axial test frame having an input comprising a linear actuator; and
a fixture configured to be attached to the uni-axial test frame, wherein the fixture is configured to facilitate a plurality of primary motions, the plurality of primary motions including a linear motion of the input and a rotational movement of an output;
an external transducer connected to the PMU and configured to collect data related to or generated by at least the plurality of primary motions;
wherein the fixture comprises an upper assembly configured to be directly or indirectly coupled to the linear actuator and a lower assembly configured to be directly or indirectly coupled to the upper assembly,
wherein the upper assembly is configured to transfer the linear motion (T_{Z}) of the linear actuator to the rotational movement (R_{X}) of the output and wherein the upper assembly and lower assembly are configured to apply an axial compression (F_{Z}) to the PMU,
wherein the fixture is configured to simultaneously provide a bending moment (M_{X}) and axial compression (F_{Z}) to the PMU,
wherein the upper assembly comprises a top surface and a pivoting platform with a connector therebetween; and
wherein the connector comprises a sleeve bearing.

7. The system of claim 6, wherein the fixture comprises a thrust bearing disposed between the linear actuator and the top surface of the upper assembly.

8. A multi-axis test system, the system comprising:
a physiological motion unit (PMU) having a first end and a second end opposite the first end;
a uni-axial test frame having an input comprising a linear actuator; and
a fixture configured to be attached to the uni-axial test frame, wherein the fixture is configured to facilitate a plurality of primary motions, the plurality of primary motions including a linear motion of the input and a rotational movement of an output;
an external transducer connected to the PMU and configured to collect data related to or generated by at least the plurality of primary motions;
wherein the fixture comprises an upper assembly configured to be directly or indirectly coupled to the linear actuator and a lower assembly configured to be directly or indirectly coupled to the upper assembly,
wherein the upper assembly is configured to transfer the linear motion (T_{Z}) of the linear actuator to the rotational movement (R_{X}) of the output and wherein the upper assembly and lower assembly are configured to apply an axial compression (F_{Z}) to the PMU,
wherein the fixture is configured to simultaneously provide a bending moment (M_{X}) and axial compression (F_{Z}) to the PMU,
wherein the upper assembly comprises a top surface and a pivoting platform with a connector therebetween, and
wherein the connector comprises a spherical bearing configured to provide three rotational degrees of freedom.

9. The system of claim 8, wherein the upper assembly is configured to transfer the linear motion (T_{Z}) of the linear actuator to the rotational movement (R_{X}) of the spherical bearing.

10. A multi-axis test system, the system comprising:
a physiological motion unit (PMU) having a first end and a second end opposite the first end;
a uni-axial test frame having an input comprising a linear actuator; and
a fixture configured to be attached to the uni-axial test frame, wherein the fixture is configured to facilitate a plurality of primary motions, the plurality of primary motions including a linear motion of the input and a rotational movement of an output;
an external transducer connected to the PMU and configured to collect data related to or generated by at least the plurality of primary motions;
wherein the fixture comprises an upper assembly configured to be directly or indirectly coupled to the linear actuator and a lower assembly configured to be directly or indirectly coupled to the upper assembly,
wherein the upper assembly is configured to transfer the linear motion (T_{Z}) of the linear actuator to the rotational movement (R_{X}) of the output and wherein the upper assembly and lower assembly are configured to apply an axial compression (F_{Z}) to the PMU,
wherein the fixture is configured to simultaneously provide a bending moment (M_{X}) and axial compression (F_{Z}) to the PMU,
wherein the upper assembly comprises a top surface and a pivoting platform with a connector therebetween;
wherein the top surface is configured to be directly or indirectly engaged by and coupled to the linear actuator and the pivoting platform is configured to be directly or indirectly engaged by and coupled to the first end of the FSU; and
wherein the lower assembly comprises at least one cable connected to the pivoting platform via a tension spring, the lower assembly further comprising at least one gear assembly configured to adjust a tension of the cable (F_{spring}).

11. The system of claim 10, wherein the axial compression (F_{Z}) is a sum of F_{spring}, an axial force (F_{OLaF}) required to produce the bending moment (M_{X}), and a force applied by the pivoting platform of the upper assembly (Fₘₐₛₛ).

12. The system of claim 10, wherein the axial compression (F_{Z}) applied to the FSU is up to 750 N.

13. The system of claim 10, wherein the lower assembly further comprises a sliding stage positionable on a plurality of linear rails for minimizing the plurality of secondary forces or moments.

14. The system of claim 13, wherein the plurality of secondary forces and moments is minimized below a predetermined threshold.

15. The system of claim 1413, wherein the plurality of secondary forces and moments is minimized below a predetermined threshold having a range of up to 5%.

16. A multi-axis test system, the system comprising:
a physiological motion unit (PMU) having a first end and a second end opposite the first end;
a uni-axial test frame having an input comprising a linear actuator; and
a fixture configured to be attached to the uni-axial test frame, wherein the fixture is configured to facilitate a plurality of primary motions, the plurality of primary motions including a linear motion of the input and a rotational movement of an output;
an external transducer connected to the PMU and configured to collect data related to or generated by at least the plurality of primary motions;
wherein the fixture comprises an upper assembly configured to be directly or indirectly coupled to the linear actuator and a lower assembly configured to be directly or indirectly coupled to the upper assembly,
wherein the upper assembly is configured to transfer the linear motion (T_{Z}) of the linear actuator to the rotational movement (R_{X}) of the output and wherein the upper assembly and lower assembly are configured to apply an axial compression (F_{Z}) to the PMU,
wherein the fixture is configured to simultaneously provide a bending moment (M_{X}) and axial compression (F_{Z}) to the PMU, and
wherein the plurality of secondary forces and moments comprises one or more of shear force (Fx, FY), and roll and yaw moment (MY, MZ) and a plurality of secondary motions comprise one or more of shear displacement (Tx, TY) and roll and yaw angle (RY, RZ), the plurality of secondary motions and the plurality of secondary forces or moments are produced by the fixture in providing the bending moment (M_{X}).

17.-21. (canceled)

22. A fixture configured to be attached to a physiological motion unit (PMU) and a uni-axial test frame having an input, the fixture comprising:
an upper assembly configured to be directly or indirectly coupled to the input to provide a bending moment (M_{X}) to the PMU;
a lower assembly configured to apply an axial compression (F_{Z}) to the PMU; and
wherein the fixture is configured to simultaneously provide the bending motion and axial compression to the PMU, and
wherein the connector comprises a spherical bearing providing three rotational degrees of freedom.

23. The fixture of claim 22, wherein the plurality of the primary motions of the upper assembly comprises the linear motion (T_{Z}) of the linear actuator and the rotational movement (R_{X}) of the spherical bearing.

24. The fixture of claim 22, wherein the upper assembly is configured to transfer the linear motion (T_{Z}) of the linear actuator to the rotational movement (R_{X}) of the spherical bearing.

25. A fixture configured to be attached to a physiological motion unit (PMU) and a uni-axial test frame having an input, the fixture comprising:
an upper assembly configured to be directly or indirectly coupled to the input to provide a bending moment (M_{X}) to the PMU, the upper assembly comprising a top surface and a pivoting platform with a connector therebetween;
a lower assembly configured to apply an axial compression (F_{Z}) to the PMU; and
wherein the fixture is configured to simultaneously provide the bending motion and axial compression to the PMU, and
wherein the lower assembly comprises at least one cable connected to the pivoting platform via a tension spring, the lower assembly further comprising at least one gear assembly configured to adjust a tension of the cable (F_{spring}).

26. The fixture of claim 25, wherein the axial compression (F_{Z}) is a sum of F_{spring}, an axial force (F_{OLaF}) required to produce the bending moment (M_{X}), and a force applied by the pivoting platform of the upper assembly (Fₘₐₛₛ).

27.-30. (canceled)

31. A method for performing a multi-axial spine test, the method comprising:
attaching a fixture of claim 16 to the uni-axial testing frame;
defining a bending profile of the PMU, the PMU comprising a functional spinal unit (FSU) and the bending profile comprising the rotational movement (R_{X}) of the spherical bearing, frequency, and number of cycles;
calculating the linear motion (T_{Z}) of the linear actuator to achieve the bending profile of the FSU;
positioning and securing the FSU between the upper assembly and the lower assembly of the fixture in a neutral position; and
applying the axial compression (F_{Z}) to the FSU.

32. A multi-axial spine testing system, the system comprising:
a physiological motion unit (PMU) having a first end and a second end opposite the first end;
a uni-axial test frame having an input comprising a linear actuator; and
a fixture configured to be attached to the uni-axial test frame, wherein the fixture is configured to facilitate a plurality of primary motions of the upper assembly, the plurality of primary motions including a linear motion of the input and a rotational movement of an output;
an external transducer connected to the PMU and configured to collect data related to or generated by at least the plurality of primary motions;
wherein the fixture comprises an upper assembly configured to be directly or indirectly coupled to the linear actuator and a lower assembly configured to be directly or indirectly coupled to the upper assembly,
wherein the upper assembly is configured to transfer the linear motion (T_{Z}) of the linear actuator to the rotational movement (R_{X}) of the output and wherein the upper assembly and lower assembly are configured to apply an axial compression (F_{Z}) to the PMU,
wherein the fixture is configured to simultaneously provide a bending moment (M_{X}) and axial compression (F_{Z}) to the PMU; and
wherein the lower assembly comprises at least one cable connected to a pivoting platform of the top assembly via a tension spring, the lower assembly further comprising at least one gear assembly configured to adjust a tension of the cable (F_{spring}).
